(19) United States
(12) Patent Application Publication
(10) Pub. No.: US 2023/0038360 A1
(43) Pub. Date: Feb. 9, 2023

(54) COMPOSITION FOR THE TREATMENT OF AMYLOID-BETA ASSOCIATED DISEASES

(71) Applicant: BASF SE, Ludwigshafen am Rhein (DE)

(72) Inventors: Ute OBERMUELLER-JEVIC, Lampertheim (DE); Stefan RUEDENAUER, Lampertheim (DE)

(21) Appl. No.: 17/786,565

(22) PCT Filed: Dec. 21, 2020
(86) PCT No.: PCT/EP2020/087423

§ 371 Date: Jun. 17, 2022

(30) Foreign Application Priority Data
Dec. 20, 2019 (EP) 19218996.7

(51) Int. Cl. A61K 31/19; A61P 25/28

(52) U.S. Cl. CPC A61K 31/19; A61P 25/28

(57) ABSTRACT

The present invention relates to compositions and methods of treatment and/or prevention of diseases and conditions associated with amyloid-beta associated diseases. In particular, the present invention relates to compositions comprising propionic acid.

The present invention relates to compositions for use in the treatment and/or prevention of amyloid-beta associated diseases. In particular, the present invention relates such compositions comprising propionic acid.

### BACKGROUND OF THE INVENTION

Amyloid-beta associated diseases, like Alzheimer's disease are neurodegenerative diseases characterized by accumulation of amyloid-beta peptides in the brain. Although these diseases have different etiologies and symptoms, they all show amyloid-beta pathology with progressive cognitive impairment and degeneration and/or death of neuron cells. So, typically amyloid-beta associated diseases are characterized by a slow onset and chronic progression. It is generally assumed that prevention of amyloid-beta pathology improves cognitive function and other symptoms of said disease.

Alzheimer's disease is the most common amyloid-beta associated disease, a chronic neurodegenerative disease characterized by progressive cognitive impairment. Eight abilities are most commonly impaired in subjects with Alzheimer's disease including memory, language, perceptual skills, attention, motor skills, orientation, problem solving and executive functional abilities. It most cases symptoms of Alzheimer disease manifest in persons over 65 years of age, although in 4 to 5% of the cases an early-onset of Alzheimer's disease is observed. Alzheimer disease affects about 6% of persons 65 years and older.

A common method of diagnosing Alzheimer's disease is based on the Alzheimer's organization's NINCDS-ADRDA Alzheimer's Criteria (2007). These criteria require that the presence of cognitive impairment and a suspected dementia syndrome be confirmed by neuropsychological testing for a clinical diagnosis of “possible or probable Alzheimer's disease”; while they need histopathologic confirmation (microscopic examination of brain tissue) for the definitive diagnosis. “Definite Alzheimer's disease” is diagnosed in case the patient meets the criteria for “probable Alzheimer's disease” and has histopathologic evidence of Alzheimer's disease via autopsy or biopsy. “Probable Alzheimer's disease” is diagnosed, if (i) dementia has been established by clinical and neuropsychological examination, (ii) cognitive impairments are progressive and present in two or more areas of cognition, (iii) the onset of the deficits has been between the ages of 40 and 90 years, and (iv) finally there must be an absence of other diseases capable of producing a dementia syndrome. “Possible Alzheimer's disease” is diagnosed if there is a dementia syndrome with an atypical onset, presentation or progression; and without a known etiology; but no co-morbid diseases capable of producing dementia are believed to be in the origin of it.

Alzheimer's disease can be classified as pre-Alzheimer's disease (or mild cognitive impairment) in its prodromal state followed by early Alzheimer's disease, moderate Alzheimer's disease, and advanced Alzheimer's disease.

Neuropsychological tests such as the mini-mental state examination (MMSE) are widely used to evaluate the cognitive impairments of a patient, in order to assist diagnosis. In the MMSE test, any score of 24 or more (out of 30) indicates a normal cognition, a score of 19 to 23 points indicates a mild cognitive impairment, a score of 10 to 18 moderate and a score of below 9 points severe cognitive impairment.

Examples of amyloid-beta associated diseases are listed in Table 1.

TABLE 1

| | |
|---|---|
| disease type | relation to amyloid-beta pathology |
| Alzheimer, familial Type 1 | alterations in APP gene |
| Alzheimer, familial Type 2 | alterations in apolipoprotein gene |
| Alzheimer, familial Type 3 | alterations in gamma-secretase gene |
| and 4 | |
| Down syndrome | increased Aβ42 production |
| hypercholesterolemia | increased Aβ42 deposits in cholesterol- |
| | rich lipid domains of membranes |
| brain traumata | unknown etiology |
| drug side effects | increased production of |
| (COX-2 inhibitors, | |
| isoprenoids) | |

A common feature in the development of amyloid-beta associated diseases like pre-Alzheimer's disease, Alzheimer's disease and Down syndrome is misfolding of proteins (proteopathy), e.g. of the amyloid-beta peptide or the tau protein in the brain, which causes an accumulation of abnormally folded amyloid-beta peptide and/or tau protein in the brain and eventually plaque formation. Amyloid-beta peptides are fragment of the larger amyloid precursor protein (APP). APP is a transmembrane protein that penetrates through neuronal membranes. APP is critical to neuron growth, survival, and post-injury repair. In Alzheimer's disease, gamma secretase and beta secretase act together in a proteolytic process which causes APP to be fragmented into smaller peptides. One of these fragments gives rise to fibrils of amyloid-beta, which then form clumps that deposit outside neurons in dense formations known as senile plaques. Accumulation of aggregated amyloid fibrils, which are believed to be the toxic form of the protein responsible for disrupting the cell's calcium ion homeostasis, induces programmed cell death (apoptosis). It is also known that amyloid-beta peptide selectively builds up in the mitochondria in the cells of Alzheimer's disease affected brains, and it also inhibits certain enzyme functions and the utilization of glucose by neurons. Various inflammatory processes and cytokines may also have a role in the pathology of amyloid-beta associated diseases. Inflammation is a general marker of tissue damage in any disease, and may be either secondary to tissue damage or a marker of an immunological response. Amyloid-beta peptide 40 and amyloid-beta peptide 42 are considered neurotoxic. Particularly amyloid-beta peptide 42 have a strong tendency to form insoluble aggregates. Usually such peptides are continually formed in normal physiologic state without deposition of aggregates. However, in pathological situations, aggregates are deposited causing said amyloid-beta associated diseases.

The cause for most Alzheimer's cases is still mostly unknown except for 1% to 5% of cases where genetic differences have been identified. Other risk factors include a history of head injuries, depression and hypertension.

The Alzheimer Association estimates that in 2019 5.8 million Americans are living with Alzheimer's disease. This number includes an estimated 5.6 million people age 65 and older and approximately 200,000 individuals under age 65 who have younger-onset Alzheimer's disease.

For treatment or prevention of amyloid beta associated diseases engagement in intellectual activities such as reading, playing games, playing musical instruments, regular social interaction are recommended as are maintenance of normal body weight, consumption of a diet rich in fruits and vegetables, regular physical exercise, limited alcohol consumption and smoking cessation. Five medications are currently used for treatment of Alzheimer's disease namely acetylcholinesterase inhibitors (tacrine, rivastigmine, galantamine and donepezil) and an NMDA receptor antagonist (memantine). The benefit from their use is small. No medication has been clearly shown to delay or halt the progression of the disease.

Although these preventive measure and treatments exist, they are often not successful. So, there is still an urgent need for compositions being suitable in the treatment and/or prevention of amyloid-beta associated diseases.

### SUMMARY OF THE INVENTION

Accordingly, the present invention provides in a first aspect a composition comprising
i) propionic acid and/or derivative(s) thereof,

for use in the treatment or prevention of an amyloid-beta associated disease, in particular wherein the propionic acid and/or derivate thereof is the only active ingredient against the amyloid-beta associated disease.

In another aspect the present invention relates to methods for treating a subject having, being suspected of having or being at risk of developing an amyloid-beta associated disease. In a further aspect the present invention relates to a nutritional supplement or a functional food comprising said composition. Another aspect of the present invention is related to the use of said composition as a nutritional supplement or a functional food, in particular in the dietary management of an amyloid-beta associated disease.

### DETAILED DESCRIPTION OF THE INVENTION

It has now been found that propionic acid or derivatives thereof can be used in the treatment or prevention of amyloid-beta associated diseases. These provide beneficial effects as described herein.

Accordingly, the present invention provides in a first aspect a composition comprising
i) propionic acid and/or derivative(s) thereof,

for use in the treatment or prevention of an amyloid-beta associated disease, of mammals, in particular wherein the propionic acid and/or derivate thereof is the only active ingredient against the amyloid-beta associated disease.

For the purpose of the present invention, the term “propionic acid and/or derivative thereof” encompasses propionic acid as well as derivatives thereof and/or any mixture thereof. Suitable derivatives are salts, esters and amides, in particular physiologically acceptable ones.

Examples for physiologically acceptable salts are alkali salts, like sodium or potassium salts, or alkaline-earth salts, like magnesium or calcium salts, or choline salts. Preferably the physiologically acceptable salts are alkali salts, in particular sodium salts or potassium salts, especially sodium salts.

Examples for physiologically acceptable esters are those derived from C1-C6 alcohols, in particular those derived from monohydric C1-C6 alcohols, e.g. those derived from methanol or ethanol, or dihydric C1-C6 alcohols, like those derived from 1,2-ethandiol, or C1-C4 alkoxy substituted monohydric alcohols, like those derived from 2-methoxyethanol, 2-ethoxyethanol or 2-butoxyethanol. Other examples for physiologically acceptable esters are glycerides, like mono-, di-, or triglycerides, in particular mono- or diglycerides. Preferably the physiologically acceptable esters are those derived from monohydric C1-C6 alcohols, e.g. those derived from methanol or ethanol, or mono- or diglycerides.

Examples for physiologically acceptable amides are those derived from mono- or di-C1-C6-alkyl amines.

In an embodiment of the present invention the propionic acid or a derivative thereof is propionic acid (=free propionic acid).

For the purpose of the present invention the term “treatment” means using an effective therapy or management to alleviate, reduce or cure the disease and/or symptoms thereof, in addition it also includes the stabilization of the disease, in order not to worsen in the course of the disease. In an embodiment of the present invention treatment is understood as using an effective therapy or management to stabilize, alleviate or reduce the disease and/or symptoms thereof.

Within the context of the present invention the term “prevention” means an effective therapy or management so that the disease does not de novo develop, manifest and/or symptoms thereof do not occur.

For the purpose of the present invention, the term “mammals” encompasses humans and non-human mammals. Examples for non-human mammals are livestock, e.g. sheep, goats, pigs, cattles, horses, camels, llamas and the like, and pets, e.g. cats, dogs and the like.

In a preferred embodiment of the present invention the composition is for use in the treatment or prevention of an amyloid-beta associated disease of humans.

In an embodiment of the present invention the composition is used for the treatment of an amyloid-beta associated disease, in particular of humans.

In another embodiment of the present invention the composition is used for the prevention of amyloid-beta associated disease, in particular of humans.

For the purpose of the present invention, the term “amyloid-beta associated disease” refers to a disease which is caused by the progressive loss of structure and/or function of neurons which may lead to the death of the affected neurons and which is caused by accumulation of amyloid beta peptides in the brain. Examples are Alzheimer's disease, Down syndrome, hypercholesterolemia, brain traumata, amyloid-beta associated disease due to drug side effects, e.g. of COX-2 inhibitors, isoprenoids etc. which cause an increased production of amyloid-beta, and the like. In particular, Alzheimer disease can be further sub-classified into Alzheimer's disease—family type 1 (caused by alteration(s) in the APP gene), Alzheimer's disease—family type 2 (caused by alteration(s) in the apolipoprotein gene) and Alzheimer's disease—family type 3 and 4 (caused by alteration(s) in the gamma secretase gene). Also, it can be differentiated between pre-, early, moderate and severe Alzheimer's disease.

In an embodiment of the present invention the composition is for use in the treatment or prevention of Alzheimer's disease, in particular of Alzheimer's disease—family type 1, Alzheimer's disease—family type 2 and/or Alzheimer's disease—family type 3 and 4, especially of humans.

In an embodiment of the present invention the composition is for use in the treatment or prevention of pre-, early, moderate or severe Alzheimer's disease, especially of humans.

In an embodiment of the present invention the composition is for use in the treatment or prevention of Down syndrome, especially of humans, in particular of symptoms attributable to the accumulation of amyloid-beta peptide(s).

In an embodiment of the present invention the composition is for use in the treatment or prevention of hypercholesterolemia, especially of humans.

In an embodiment of the present invention the composition is for use in the treatment or prevention of amyloid-beta associated diseases due to drug side effects, e.g. of COX-2 inhibitors, isoprenoids etc, especially of humans.

Human subjects with Alzheimer's disease show often at least one of the following symptoms: memory impairment, language problems, personality changes, confusion, hallucinations and delusions, loss of the ability to perform activities of the daily live.

In an embodiment of the present invention the composition is for use in the treatment or prevention of memory impairment, e.g. of a subject having or being suspected or at risk of developing Alzheimer's disease.

In another embodiment of the present invention the composition is for use in the treatment or prevention of personality changes, e.g. of a subject having or being suspected or at risk of developing Alzheimer's disease.

In another embodiment of the present invention the composition is for use in the treatment or prevention of language problems, e.g. of a subject having or being suspected or at risk of developing Alzheimer's disease.

In another embodiment of the present invention the composition is for use in the treatment or prevention of confusion, e.g. of a subject having or being suspected or at risk of developing Alzheimer's disease.

In another embodiment of the present invention the composition is for use in the treatment or prevention of hallucinations and/or delusions, e.g. of a subject having or being suspected or at risk of developing Alzheimer's disease.

In another embodiment of the present invention the composition is for use in the treatment or prevention of the loss of the ability to perform activities of daily life, e.g. of a subject having or being suspected or at risk of developing Alzheimer's disease.

In clinical practice the Mini-Mental-State-Test (MMSE) has been developed which may at least partly be used or assist in the diagnosis of Alzheimer's disease.

In another embodiment of the present invention the composition for use in the treatment or prevention of an amyloid-beta associated disease can further comprise one or more vitamins or related compounds thereto. Examples of vitamins and related compounds thereto include vitamin A (e.g. retinol, retinyl acetate, retinyl palmitate, retinyl stearate, retinyl esters with other long-chain unsaturated fatty acids, retinal, retinoic acid and the like), vitamin B1 (e.g. thiamine, thiamine pyrophosphate, TPP, thiamine triphosphate, TTP, thiamine hydrochloride, thiamine mononitrate and the like), vitamin B2 (e.g. riboflavine, flavine mononucleotide (FMN), flavine adenine dinucleotide (FAD), lactoflavine, ovoflavine and the like), vitamin B3 (e.g. nicotinic acid, nicotinamide, nicotinamide adenine dinucleotide (NAD), nicotinic acid mononucleotide (NicMN), pyridine-3-carboxylic acid and the like, as well as the vitamin B3-precursor tryptophan), pantothenic acid (e.g. pantothenate, panthenol and the like), vitamin B6 (e.g. pyridoxine, pyridoxal, pyridoxamine, pyridoxine hydrochloride and the like), biotin, folic acid (e.g. folate, folacin, pteroylglutamic acid and the like), vitamin B12 (e.g. cobalamin, methylcobalamin. deoxyadenosylcobalamin, cyanocobalamin, hydroxycobalamin, adenosylcobalamin and the like), vitamin E (e.g. alpha-, beta-, gamma- and/or delta-tocopherol, alpha-, beta-, gamma- and/or delta-tocopherol acetate, alpha-, beta-, gamma- and/or delta-tocopherol succinate, alpha-, beta-, gamma- and/or delta-tocopherol nicotinate, alpha-, beta-, gamma- and/or delta tocotrienol and the like), vitamin K (e.g. vitamin K1, phylloquinone, naphthoquinone, vitamin K2, menaquinone-7, vitamin K3, menaquinone-4, menadione, menaquinone-8, menaquinone-8H, menaquinone-9, menaquinone-9H, menaquinone-10, menaquinone-11, menaquinone-12, menaquinone-13 and the like), vitamin C (ascorbic acid), vitamin D (e.g. calciferol, cholecalciferol, 1,25-dihydroxyvitamin D, ergocaliferol and the like), and the like and/or mixtures thereof. The presence and amounts of specific vitamins and/or related compounds thereto will vary depending on the intended use.

In another embodiment of the present invention the composition for use in the treatment or prevention of an amyloid-beta associated disease does not comprise one or more vitamins or related compounds thereto.

In another embodiment of the present invention the composition for use in the treatment or prevention of an amyloid-beta associated disease can further comprise one or more carotenoids. Examples of carotenoids include astaxanthin, alpha-carotene, beta-carotene, beta-cryptoxanthin, lutein, lycopene, meso-zeaxanthin, zeaxanthin and the like (including cis/trans isomers) and/or mixtures thereof. The presence and amounts of specific carotenoids will vary depending on the intended use.

In another embodiment of the present invention the composition for use in the treatment or prevention of an amyloid-beta associated disease does not comprise one or more carotenoids.

In another embodiment of the present invention the composition for use in the treatment or prevention of an amyloid-beta associated disease can further comprise one or more medium-chain fatty acids. These medium-chain fatty acids may be provided as free fatty acids, as glycerides (e.g. as monoglycerides, diglycerides or triglycerides and/or as mixtures thereof), as phospholipids, as alkyl esters and/or as mixtures thereof, preferably as glycerides, more preferably as triglycerides or alkyl esters. Examples of medium-chain fatty acids include caproic acid, caprylic acid, capric acid, lauric acid and the like and/or mixtures thereof. The presence and amounts of specific medium chain fatty acids will vary depending on the intended use.

In another embodiment of the present invention the composition for use in the treatment or prevention of an amyloid-beta associated disease does not comprise one or more medium-chain fatty acids.

In another embodiment of the present invention the composition for use in the treatment or prevention of an amyloid-beta associated disease can further comprise one or more long-chain fatty acids. These long-chain fatty acids may be provided as free fatty acids, as glycerides (e.g. as monoglycerides, diglycerides or triglycerides and/or as mixtures thereof), as phospholipids, as alkyl esters and/or as mixtures thereof, preferably as glycerides, more preferably as triglycerides or as alkyl esters. Examples of long chain fatty acids include saturated long chain fatty acids (e.g. myristic acid, palmitic acid, stearic acid, arachidic acid, behenic acid, lignoceric acid, cerotic acid and the like and/or mixtures thereof), mono-unsaturated long chain fatty acids (e.g. myristoleic acid, palmitoleic acid, sapienic acid, oleic acid, elaidic acid, vaccenic acid, erucic acid and the like and/or mixtures thereof), polyunsaturated long chain fatty acids (e.g. linoleic acid, linoelaidic acid, alpha-linolenic acid, arachidonic acid, eicosapentaenoic acid, docosapentenoic acid, docosahexaenoic acid and the like and/or mixtures thereof) and/or mixtures thereof. These long chain fatty acids are comprised for example in vegetable oils, single cell oils and marine oils, e.g. fish oil, krill oil and the like. The presence and amounts of specific long chain fatty acids will vary depending on the intended use.

In another embodiment of the present invention the composition for use in the treatment or prevention of an amyloid-beta associated disease does not comprise one or more long-chain fatty acids.

In another embodiment of the present invention the composition for use in the treatment or prevention of an amyloid-beta associated disease can further comprise one or more prebiotics other than human milk oligosaccharides (HMOs). Examples of prebiotics include water-insoluble fibers (e.g. lignin, cellulose, hemi-cellulose, resistant starch, xanthum gum and the like and/or mixtures thereof), water-soluble fibers (e.g. arabinoxylan, inulin, pectin, alginic acid and derivatives thereof, agar, carrageen, raffinose, xylose, polydextrose, lactulose and the like and/or mixtures thereof), other oligosaccharides like xylooligosaccharides, fructooligosaccharides, galactooligosaccharides, isomalto-oligosaccharides and the like and/or mixtures thereof, in particular water-insoluble fibers (e.g. lignin, cellulose, hemi-cellulose, resistant starch, xanthum gum and the like and/or mixtures thereof), water-soluble fibers (e.g. arabinoxylan, inulin, pectin, alginic acid and derivatives thereof, agar, carrageen, raffinose, xylose, polydextrose, lactulose and the like and/or mixtures thereof) and the like and/or mixtures thereof. The presence and amounts of specific prebiotics will vary depending on the intended use.

In another embodiment of the present invention the composition for use in the treatment or prevention of an amyloid-beta associated disease does not comprise one or more prebiotics.

In another embodiment of the present invention the composition for use in the treatment or prevention of an amyloid-beta associated disease can further comprise one or more probiotics. Examples of probiotics optionally present in the composition of the present invention include microorganisms or parts thereof of the family Lactobacillaceae, e.g. of the genus Lactobacillus (e.g. the species Lactobacillus acidophilus, Lactobacillus alimentarius, Lactobacillus casei, Lactobacillus delbrueckii (like Lactobacillus delbrueckii spp. bulgaricus, Lactobacillus delbrueckii spp. delbrueckii, Lactobacillus delbrueckii spp. lactis), Lactobacillus helveticus, Lactobacillus plantarum, Lactobacillus reuteri, Lactobacillus rhamnosus, Lactobacillus salivarius and the like), of the genus Bifidobacterium (e.g. the species Bifidobacterium animalis, Bifidobacterium bifidum, Bifidobacterium breve, Bifidobacterium infantis, Bifidobacterium lactis, Bifidobacterium longum and the like), of the genus Pediococcus (e.g. the species Pediococcus acidilactici, Pediococcus pentosaceus and the like), of the genus Lactococcus (e.g. the species Lactococcus lactis (like Lactococcus latis spp. cremoris, Lactococcus lactis spp. lactic and the like) and of the genus Streptococcus (e.g. the species Streptococcus thermophilus and the like) and of the genus Faecalibacterium (e.g. the species Faecalibacterium prausnitzii) and of the genus Bacillus (e.g. the species Bacillus subtilis) and the like and/or mixtures thereof. The presence and amounts of specific probiotics will vary depending on the intended use.

In another embodiment of the present invention the composition for use in the treatment or prevention of an amyloid-beta associated disease does not comprise one or more probiotics.

In another embodiment of the present invention the composition for use in the treatment or prevention of an amyloid-beta associated disease can further comprise one or more phenolic compounds. Examples of phenolic compounds include monophenols (e.g. apiole, carnosol, carvacrol, dillapiole, rosemarinol and the like), flavonoids (e.g. quercetin, kaempferol, myricetin, fisetin, rutin, isorhamnetin, hesperidin, naringenin, silybin, eriodyctiol, acacetin, apigenin, chrysin, diosmetin, tangeritin, luteolin, catechins like epigallocatechin gallate, theaflavin, thearubigins, proanthocyanidins, pelargonidin, peonidin, cyanidin, delphinidin, malvidin, petunidin and the like), isoflavonoids (e.g. daidzein, genistein, glycitein and the like), aurones, chalconoids, flavonolignans, lignans, phytoestrogens, stilbenoids (e.g. resveratrol, pterostilbene, piceatannol and the like), curcuminoids (e.g. curcumin and the like), tannins, aromatic acids (e.g. salicylic acid, vanillic acid, gallic acid, ellagic acid, tannic acid, caffeic acid, chlorogenic acid, cinnamic acid, ferulic acid, coumarin and the like), phenylethanoids (e.g. tyrosol, hydroxytyrosol, oleocanthal, oleuropein and the like), capsaicin, gingerol, alkylresorcinol and the like and/or mixtures thereof. The presence and amounts of specific phenolic compounds will vary depending on the intended use.

In another embodiment of the present invention the composition for use in the treatment or prevention of an amyloid-beta associated disease does not comprise one or more phenolic compounds.

In another embodiment of the present invention the composition for use in the treatment or prevention of an amyloid-beta associated disease can further comprise one or more herbals, e.g. known from Chinese diets, Indian diets, Mediterranean diets and the like. These herbals can be provided as powders obtained from the plant and/or fungus or parts thereof or as extracts thereof.

Examples for herbals known from Chinese diets include extracts or powders of hawthorn fruit, wolfberry, spatholobus stem, caterpillar fungus, cloud mushroom, crysanthemum, honeysuckle flower, mulberry leaf, glossy privet fruit, malaytea scurfpea fruit, cherokee rose fruit, palmleaf raspberry fruit, Chinese magnoliavine fruit, reishi mushroom, ephedra, epimedium, Angelica root, Astragalus root, rhubarb, licorice, morinda root, notoginseng, white peony root, American ginseng, fleeceflower root, kudzu root, rehmannia root, salvia root, Chinese yam, wild buckwheat rhizome, tall gastrodia tuber, golden root, Cassia seed, Coix seed, Dodder seed and the like and/or mixtures thereof.

Examples for herbals known from Indian diets include extracts or powders of Amalaki (Indian gooseberry), Haritaki (chebulic myrobalan), Bibhitaki (beleric), Haldi (turmeric), Tulsi (holy basil), Shigru (moringa), Twak (cinnamon), Yashtimadhu (licorice root), Dhanyaka (coriander), Ashwagandha (winter cherry), Kumkuma (saffron), Manjistha (Indian madder), Brahmi (bacopa), Neem (margosa), Ajwain (Bishop's weed), Elaichi (cardamom), Shikakai (Acacia concinna), Shatavari (wild asparagus), Jeera (cumin), Guduchi (tinospora) and the like and/or mixtures thereof. Examples for herbals known from Mediterranean diets include extracts or powders of rosemary, basil, parsley, saffron, thyme, oregano, sage, cilantro, lemon, orange, grape, grapeseed, fig, blueberry, raspberry, strawberry, cherry, fennel, sesame seeds, pine seeds, garlic, onion, ginger root, pepper, chili and the like, olive oil and/or mixtures thereof.

The presence and amounts of specific herbals will vary depending on the intended use.

In another embodiment of the present invention the composition for use in the treatment or prevention of an amyloid-beta associated disease does not comprise one or more herbals.

In another embodiment of the present invention the composition for use in the treatment or prevention of an amyloid-beta associated disease can further comprise one or more minerals. Examples of minerals include such ones comprising calcium, phosphorous, iodine, iron, magnesium, copper, zinc, manganese, chlorine, potassium, sodium, selenium, chromium, molybdenum and the like and/or mixtures thereof. Minerals are usually added in salt form. The presence and amounts of specific minerals will vary depending on the intended population.

In another embodiment of the present invention the composition for use in the treatment or prevention of an amyloid-beta associated disease does not comprise one or more minerals.

In another embodiment of the present invention the total amount of the propionic acid and/or derivative(s) thereof is from 10 to 100 wt % of the total composition, preferably from 50 to 100 wt %.

In another embodiment of the present invention the total amount of the propionic acid and/or derivative(s) thereof is about 100 wt % of the total composition.

In another embodiment the total amount of the propionic acid and/or derivative(s) thereof is from 5 to 99.9 wt % of the total composition, in particular from 10 to 99 wt %, preferably from 15 to 90 wt %, more preferably from 20 to 85 wt %, even more preferably from 25 to 75 wt %. In yet another embodiment the total amount of the propionic acid or derivative(s) thereof is from 90 to 99.9 wt % of the total composition, preferably from 95 to 99 wt %, more preferably from 95 to 98 wt %. In yet another embodiment the total amount of the propionic acid or derivative(s) thereof is from 50 to 90 wt % of the total composition, preferably from 55 to 80 wt %, more preferably from 60 to 75 wt %.

The compositions for use in the treatment or prevention of an amyloid-beta associated disease of the present invention can be prepared by known methods. E.g. in case the composition comprises more components by mixing the propionic acid and/or derivative(s) thereof with the optionally further components e.g. vitamins and related compounds thereto, carotenoids, medium chain fatty acids, long chain fatty acids, prebiotics, probiotics, phenolic compounds, herbals, minerals and the like and/or mixtures thereof, as known in the art.

The compositions of the present invention for use in the treatment or prevention of amyloid-beta associated diseases can be administered orally, enterally or parenterally, preferably orally.

In an embodiment of the present invention the composition for use in the treatment or prevention of amyloid-beta associated diseases is an orally administrable composition.

Furthermore, the present invention provides a method to treat a subject having an amyloid-beta associated disease, or to prevent a subject being suspected of having or being at risk of developing an amyloid-beta associated diseases, by administering to the subject an effective amount of the composition which comprises
i) propionic acid and/or derivative(s) thereof,

in particular wherein the propionic acid or derivate thereof is the only active ingredient against the amyloid-beta associated disease.

In another embodiment thereof, the daily application rate of the propionic acid and/or derivative(s) thereof is from 0.1 to 20.0 g, preferably from 1.0 to 15.0 g, even more preferably from 2.0 to 8.0 g.

In another embodiment thereof, the propionic acid and/or a derivative thereof can be administered in an application rate from 0.1 to 8.0 g, preferably from 0.1 to 6.0 g/day, even more preferably from 0.5 to 5.0 g.

In another embodiment thereof the daily application rate of the propionic acid and/or derivative(s) thereof is from 4.0 to 15.0 g, preferably from 5.0 to 12.0 g, even more preferably from 6.0 to 10.0 g.

In another embodiment of the invention said subject is a human, preferably in the age of 12 years or older, more preferably 18 years or older, even more preferably 35 years or older, in particular 50 years or older, even more in particular 60 years or older.

Furthermore, the present invention provides the use of a composition comprising
i) propionic acid and/or derivative(s) thereof,

for the manufacture of a medicament for the treatment or prevention of amyloid-beta associated diseases.

Furthermore, the present invention provides a nutritional supplement or a functional food comprising a composition which comprises
i) propionic acid or derivative(s) thereof,

for use in the dietary management of as subject having, being suspected of having or being at risk of developing of an amyloid-beta associated disease, in particular wherein the propionic acid or derivate thereof is the only active ingredient against the amyloid-beta associated disease.

For the purpose of the present invention the term “nutritional supplement” means a manufactured product intended to supplement a diet, in particular of subjects having, being suspected of having or being at risk of developing amyloid-beta associated diseases. Examples for nutritional supplements include “dietary supplements” and “medical foods”. A dietary supplement is intended to supplement a diet, in particular of subjects having, being suspected of having or being at risk of developing amyloid-beta associated diseases, however it needs not to be used under medical supervision. A medical food is also intended to supplement a diet, in particular of subjects having, being suspected of having or being at risk of developing amyloid-beta associated diseases, but it is under medical supervision. The terms “medical foods” and “food for special medical purpose” are interchangeable.

In an embodiment of the present invention the nutritional supplement is a dietary supplement.

In another embodiment of the present invention the nutritional supplement is a medical food.

In another embodiment thereof, the daily application rate of the propionic acid and/or derivative(s) thereof is from 0.1 to 20.0 g, preferably from 1.0 to 15.0 g, even more preferably from 2.0 to 8.0 g.

In another embodiment thereof, the daily application rate of propionic acid and/or derivative(s) thereof is 4.0 to 15.0 g, preferably from 5.0 to 12.0 g, even more preferably from 6.0 to 10.0 g.

Preferably, propionic acid and/or a derivative thereof can be administered in an application rate from 0.1 to 8.0 g, preferably from 0.1 to 6.0 g/day, even more preferably from 0.5 to 5.0 g.

Furthermore, the present invention provides a method for the dietary management of a subject having, being suspected of having or being at risk of developing amyloid-beta associated diseases and/or associated morbidities thereto, by administering to the subject an effective amount of the nutritional supplement or of the functional food which comprises a composition comprising
i) propionic acid and/or derivative(s) thereof,

in particular wherein the propionic acid or derivate thereof is the only active ingredient against the amyloid-beta associated disease.

In another embodiment of the invention said subject is a human, preferably in the age of 12 years or older, more preferably 18 years or older, even more preferably 35 years or older, in particular 50 years or older, even more in particular 60 years or older.

Both, the medicament for the respective specific use and the nutritional supplement of the present invention can be delivered in any suitable format. Formulations suitable for oral administration may be in the form of capsules, tablets, pills, dragees, lozenges (using a flavored basis, usually sucrose and acacia or tragacanth), powders, granules, and the like or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each comprising the predetermined amount of propionic acid and/or derivative(s) thereof and optional further components. The medicament and/or the nutritional supplement of the present invention may also be administered as a bolus, electuary or paste.

In solid dosage forms for oral administration (capsules, tablets, pills, dragees, lozenges, powders, granules, and the like), the desired components of the composition may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) coloring agents; and (11) flavors, such as lemon, orange, apricot, banana, cherry, strawberry, raspberry, blueberry, peppermint, vanilla, chocolate, coffee, cappuccino flavor and the like. In some cases, the compositions may also comprise buffering agents.

In a specific embodiment, powders and/or granules can be reconstituted with water or another aqueous liquid prior to consumption.

For the purpose of the present invention the term “functional food” means a food which is fortified with the composition according to the present invention and intended to be used in a diet, in particular of subjects having, being suspected of having or being at risk of developing amyloid-beta associated diseases. The terms “functional food” and “fortified food” are interchangeable.

Examples for foods being suitable for the preparation of functional foods are (1) dairy products e.g. yogurt, dessert, smoothie, milk and the like or mixtures thereof; (2) bakery products e.g. bread, rolls, pasta, cookie, cake, cereal bar and the like or mixtures thereof; (3) candy products e.g. candies, gummies, chewing gum, chocolate, pudding, cookie and the like or mixtures thereof; (4) beverage products e.g. fruit juice, vegetable juice, lemonade, water and the like or mixtures thereof.

In an embodiment of the present invention the functional food is for use in the dietary management of subjects having, being suspected of having or being at risk of developing amyloid-beta associated diseases.

In another embodiment thereof, the daily application rate of the at least one propionic acid or derivative(s) thereof is 0.1 to 20.0 g, preferably from 1.0 to 15.0 g, even more preferably from 2.0 to 8.0 g. In another embodiment thereof the daily application rate of the propionic acid and/or derivative(s) thereof is from 4.0 to 15.0 g, preferably from 5.0 to 12.0 g, even more preferably from 6.0 to 10.0 g. In another embodiment the propionic acid and/or a derivative thereof can be administered in an application rate from 0.1 to 8.0 g, preferably from 0.1 to 6.0 g/day, even more preferably from 0.5 to 5.0 g.

Furthermore, the present invention provides a method for the dietary management of a subject having, being suspected to have or being at risk of developing amyloid-beta associated diseases, by administering to the subject an effective amount of the functional food which comprises a composition comprising
i) propionic acid and/or derivative(s) thereof,

in particular wherein the propionic acid or derivate thereof is the only active ingredient against the amyloid-beta associated disease.

In another embodiment of the invention said subject is a human, preferably in the age of 12 years or older, more preferably 18 years or older, even more preferably 35 years or older, in particular 50 years or older, even more in particular 60 years or older.

The functional food of the present invention can be prepared by known techniques and it can have any suitable type of format such as (1) a dairy product e.g. yogurt, dessert, smoothie, milk and the like or mixtures thereof; (2) a bakery product e.g. bread, rolls, pasta, cookie, cake, cereal bar and the like or mixtures thereof; (3) a candy product e.g. candies, gummies, chewing gum, chocolate, pudding, cookie and the like or mixtures thereof; (4) a beverage product e.g. fruit juice, vegetable juice, lemonade, water and the like or mixtures thereof.

In an embodiment of the present invention the nutritional supplement or the functional food are for use in the dietary management of Alzheimer's disease, in particular of Alzheimer's disease—family type 1, Alzheimer's disease—family type 2 and/or Alzheimer's disease—family type 3 and 4, especially of humans.

In an embodiment of the present invention the nutritional supplement or the functional food are for use in the dietary management of pre-, early, moderate or severe Alzheimer's disease, especially of humans.

In an embodiment of the present invention the nutritional supplement or the functional food are for use in the dietary management of Down syndrome, especially of humans.

In an embodiment of the present invention the nutritional supplement or the functional food are for use in the dietary management of hypercholesterolemia, especially of humans.

In an embodiment of the present invention the nutritional supplement or the functional food are for use in the dietary management of amyloid-beta associated diseases due to drug side effects, e.g. of COX-2 inhibitors, isoprenoids etc, especially of humans.

Furthermore, it is contemplated herein that the composition for use in the treatment or prevention of amyloid-beta associated diseases, the nutritional supplement and the functional food, in particular for use in the dietary management of amyloid-beta associated diseases, respectively, as disclosed herein, can be co-administered to subjects receiving at least one pharmaceutical against said amyloid-beta associated diseases. Examples for pharmaceuticals used in the treatment of amyloid-beta associated diseases are acetylcholinesterase inhibitors, e.g. tacrine, rivastigmine, galantamine and donepezil, and NMDA receptor antagonist, e.g. memantine.

Furthermore, the present invention provides a method to treat a subject having an amyloid-beta associated disease, by administering to the subject
a) an effective amount of a composition for use in the treatment or prevention of amyloid-beta associated diseases, a nutritional supplement or a functional food, in particular for use in the dietary management of amyloid-beta associated diseases, which comprises
   i) propionic acid and/or derivative(s) thereof, and
b) an effective amount of at least one pharmaceutical suitable to treat said amyloid-beta associated diseases,

in particular, wherein the application rate of the at least one pharmaceutical suitable to treat said amyloid-beta associated diseases is reduced compared to a treatment with the at least one pharmaceutical alone.

In an embodiment thereof the pharmaceutical suitable to treat said amyloid-beta associated disease is a pharmaceutical improving the degradation of beta-amyloid (e.g. aducanumab), a pharmaceutical inhibiting the formation of plaques (e.g. solanezumab, or Na-cromolyn +ibuprufen), a pharmaceutical improving the degradation of plaques (e.g. gantenerumab), an acetylcholinesterase inhibitor (e.g. tacrine, rivastigmine, galantamine donepezil or octohydroaminoacridinsuccinat), an NMDA receptor antagonist (e.g. memantine), or another (e.g. amilomotid, masitinib, leuko-methylthionium, nilvadipin, trigrilzol, lecanemab, levetiracetam or blarcamesin). In an embodiment thereof the method does not include the use of a HMO.

Furthermore, the present invention provides a composition comprising

a) propionic acid and/or derivative(s) thereof, and

b) at least one pharmaceutical suitable to treat said amyloid-beta associated diseases, in particular for use as a medicament, especially for use in the treatment or prevention of an amyloid-beta associated disease.

In an embodiment thereof the pharmaceutical suitable to treat said amyloid-beta associated disease is a pharmaceutical improving the degradation of beta-amyloid (e.g. aducanumab), a pharmaceutical inhibiting the formation of plaques (e.g. solanezumab, or Na-cromolyn +ibuprufen), a pharmaceutical improving the degradation of plaques (e.g. gantenerumab), an acetylcholinesterase inhibitor (e.g. tacrine, rivastigmine, galantamine donepezil or octohydroaminoacridinsuccinat), an NMDA receptor antagonist (e.g. memantine), or another (e.g. amilomotid, masitinib, leuko-methylthionium, nilvadipin, trigrilzol, lecanemab, levetiracetam or blarcamesin). In an embodiment thereof the composition does not include a HMO.

Within the context of the present application, it is noted that in case a composition comprises several components that these can also be administered together or separately, as the case may be.

Also, within the context of the present invention, ratios given are weight to weight ratios unless stated otherwise. In addition, in the context of the invention, the terms “comprising” or “comprises” do not exclude other possible elements. The composition of the present invention, including the embodiments described herein, can comprise, consist of, or consist essentially of the essential elements and limitations of the invention described herein, as well as any additional or optional ingredients, components, or limitations described herein or otherwise depending on the needs. The terminology as set forth herein is for description of the embodiments only and should not be construed as limiting the disclosure as a whole. Furthermore, as used in the description and the appended claims, the singular forms “a”, “an”, and “the” are inclusive of their plural forms, and the other way around, unless the context clearly indicates otherwise. It is to be understood that the embodiments of the subject matter of the invention can be applied in the specific context but also in other combinations, without leaving the scope of the invention. E.g. it is understood that the embodiments mentioned for the composition of the present invention also apply for composition for use as a medicament, etc. The same applies for the respective conditions and/or diseases.

### EXAMPLES

The following examples illustrate certain exemplary embodiments of the subject matter of the present invention as detailed above. The Examples are given solely for the purpose of illustration and are not to be construed as limitation.

### Example 1

### In Vivo Study on the Effects of Sodium Propionate on Amyloid-Beta Induced Cell Effects in C. Elegans

The biological model C. elegans was used to study the effects of sodium propionate on amyloid-beta associated pathology. C. elegans has become an excellent and well recognized model for studying neurodegenerative diseases (Zhou et al., 2011; Hekimi et al., 2011; Kaletsky & Murphy, 2010). This nematode contains 302 neuron cells with neurotransmitters and neuropeptides similar to the mammalian nervous system. All the basic functions in the worm such as development, feeding and movement are controlled by the nervous system. The model is particularly useful for studying amyloid-beta associated pathologies, since transgenic strains have been generated able to express the human Aβ1-42 peptide in muscle cells, producing a progressive paralysis in the nematodes as a result of toxic effects (Link, 1995). Therefore, this model using such strains is useful to study substances in view of their ability to reduce β-amyloid peptide cell toxicity.

In the present experiment, the transgenic C. elegans strain CL4176 expressing amyloid-beta peptide in muscle cells was used. The worms were cultured in plates with nematode growth medium (control) or in plates supplemented with different concentrations of sodium propionate (test substance). The expression of Aβ1-42 peptide was induced in the nematodes by temperature control, and the percentage of worms paralyzed was determined at different time points. Experiments were performed in duplicate.

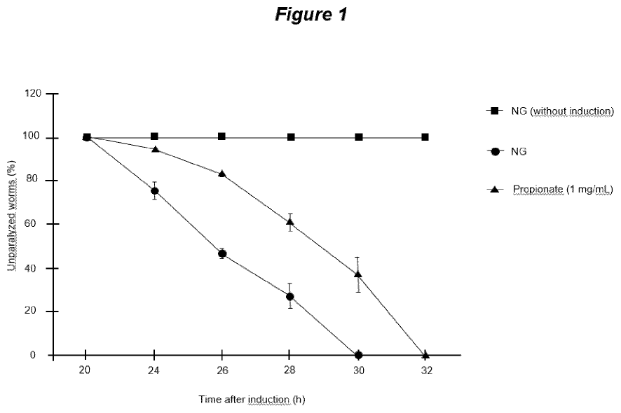

At an application rate of 1 mg/ml sodium propionate the number of paralyzed C. elegans is significantly lower compared to the untreated (however Aβ1-42 peptide expressing) C. elegans after 24 hours. This clearly shows that sodium propionate protects against the negative results of Aβ1-42 peptide expression. The results are shown in FIG. 1.

1.-6. (canceled)

7. A composition comprising
i) propionic acid and/or derivative(s) thereof,
for use in the treatment and/or prevention of an amyloid-beta associated disease.

8. The composition according to claim 7, wherein the amyloid-beta associated disease is Alzheimer's disease, Down syndrome, hypercholesterolemia, brain traumata or an amyloid-beta associated disease due to drug side effects of COX-2 inhibitors and/or isoprenoids.

9. A method for treating a subject having, suspected of having or being at risk of developing amyloid-beta associated diseases, as, comprising administering to the subject an effective amount of a composition comprising
i) propionic acid and/or derivative(s) thereof.

10. A nutritional supplement or a functional food comprising a composition comprising
i) propionic acid and/or derivative(s) thereof,
for use in the dietary management of an of amyloid-beta associated disease.

11. The nutritional supplement or the functional food according to claim 10, wherein the amyloid-beta associated diseases is Alzheimer's disease, Down syndrome, hypercholesterolemia, brain traumata or an amyloid-beta associated disease due to drug side effects of COX-2 inhibitors and/or isoprenoids.

12. A method for the dietary management of a subject having, suspected of having or being at risk of developing an amyloid-beta associated disease, comprising administering to the subject an effective amount of a composition comprising
i) propionic acid and/or derivative(s) thereof.
